(19) United States
(12) Patent Application Publication
(10) Pub. No.: US 2019/0070240 A1
(43) Pub. Date: Mar. 7, 2019

(54) GYNOSTEMMA PENTAPHYLLUM-CONTAINING HEALTH CARE PHARMACEUTICAL COMPOSITION FOR PREVENTING HYPERLIPIDEMIA, HYPERTENSION, HYPERGLYCEMIA AND GOUT

(71) Applicants: Shaoke WANG, Ankang (CN); Theresa Y Y TSE, Hongkong (CN); Zhao ZHANG, Ankang (CN)

(72) Inventors: Shaoke WANG, Colorado Springs, CO (US); Theresa Y Y TSE, Hongkong (CN); Zhao ZHANG, Ankang (CN); Ping DOU, Colorado Springs, CO (US); Jingrui XU, Ankang (CN); Hongwei TIAN, Ankang (CN); Wanyu ZHANG, Ankang (CN)

(73) Assignees: Shaoke WANG; Qirun XIE; Zhao ZHANG

(21) Appl. No.: 16/184,286

(22) Filed: Nov. 8, 2018

Related U.S. Application Data
(63) Continuation of application No. PCT/CN2017/082122, filed on Apr. 27, 2017.

(30) Foreign Application Priority Data
May 9, 2016 (CN) 201610300713.9

(51) Int. Cl. A61K 36/424; A61K 36/46; A61K 36/605; A61K 36/68; A61K 36/899; A61K 36/488; A61K 36/185; A61K 36/739; A61P 19/02

(52) U.S. Cl. CPC A61K 36/424; A61K 36/46; A61K 36/605; A61K 36/68; A61P 19/02; A61K 36/488; A61K 36/185; A61K 36/739; A61K 36/899

(57) ABSTRACT

A gynostemma pentaphyllum-containing health care pharmaceutical composition for preventing hyperlipidemia, hypertension, hyperglycemia and gout is prepared by mixing raw materials of gynostemma pentaphyllum, folium eucommiae, folium mori, semen plantaginis, herba lophatheri, pueraria mirifica powder, pelargonium graveolens and sanguisorba minor, and the raw materials are in percentage by mass: 40-70% of gynostemma pentaphyllum, 5-20% of folium eucommiae, 5-20% of folium mori, 5-10% of semen plantaginis, 2-10% of herba lophatheri, 2-10% of pueraria mirifica powder, 2-8% of pelargonium graveolens and 2-8% of sanguisorba minor. The gynostemma pentaphyllum is prepared by mixing diploid gynostemma pentaphyllum leaves and tetraploid gynostemma pentaphyllum leaves according to a ratio. The pharmaceutical composition is mainly aimed at people who suffer from hyperlipidemia, hypertension, hyperglycemia and hyperuricemia, and used for preventing and treating the “four hypers

### CROSS-REFERENCE TO RELATED APPLICATIONS

This application is a continuation of International Patent Application No. PCT/CN2017/082122 with a filing date of Apr. 27, 2017, designating the United States, now pending, and further claims priority to Chinese Patent Application No. 2016103007139 with a filing date of May 9, 2016. The content of the aforementioned applications, including any intervening amendments thereto, are incorporated herein by reference.

### FIELD OF THE INVENTION

The present disclosure belongs to the technical field of traditional Chinese medicine, and particularly relates to a gynostemma pentaphyllum-containing health care pharmaceutical composition for preventing hyperlipidemia, hypertension, hyperglycemia and gout.

### BACKGROUND OF THE INVENTION

Hyperlipidemia, hypertension, hyperglycemia are commonly known as “three hypers” and are serious problems that influence human health in China at present. Hundreds of millions of people have these problems throughout the country, and the number is increased year by year. Such serious problem still exist everywhere in many developed countries internationally, and also belong to a medical problem. The “three hypers” is a source of many human diseases and sub-health. Along with development and deterioration of the “three hypers”, a series of symptoms and complications may be caused, and human health or even lifetime may be seriously influenced.

The most important biochemical basis of gout is hyperuricemia. In recent years, increasingly popular “four hypers” medically refer to hyperlipidemia, hypertension, hyperglycemia and hyperuricemia. Researches show that, “four hypers” will be epidemic in large scale in China within 20 years. According to survey data announced by the Ministry of Health that a hypertension prevalence rate of adults is 18.8%, a diabetes prevalence rate of adults is 2.6% and a dyslipidemia prevalence rate of adults is 18.6%. China will face large-scale epidemic of the hyperlipidemia, hypertension, hyperglycemia and hyperuricemia in the future 20 years.

At present, the “four hypers” are mainly prevented and treated with Western medicines in medical field. These medicines need to be taken for a long time or even the whole life, causing great expense and certain side effects. Ideal means for prevention and treatment of the “four hypers” may be early prevention and early intervention by taking a comprehensive formula of heath care plants as a main mode under the cooperation of a good living habit. In this way, this means is safe and effective and does not have any side effect, and people may be more healthy by virtue of tonic plants.

### SUMMARY OF THE INVENTION

In order to solve the above problems, the present invention provides a gynostemma pentaphyllum-containing health care pharmaceutical composition for preventing hyperlipidemia, hypertension, hyperglycemia and gout. Dirt, wastes and toxins on inner walls of blood are removed, so that the blood is unobstructed in blood vessels, and blood vessels of the heart, brain, kidney, liver and other organs may be softened; and therefore senescent cells are eliminated from the human body, and self-regulation of the human body restores to be normal.

The gynostemma pentaphyllum is a cucurbitaceous gynostemma perennial herbaceous vine. 16 kinds of 3 subspecies are discovered around the world, 11 kinds of 1 subspecies are discovered in China, and 6 subspecies are discovered in Pingli (gynostemma aureobasidium, gynostemma longipes, fiveleaf gynostemma, white-hair gynostemma, gynostemma laxum, sweet gynostemma and gynostemma yixingense). The gynostemma pentaphyllum is mainly distributed in the south of Qinling mountain and various provinces and regions of the Yangtze River Basin. The gynostemma pentaphyllum is preferred to environments of rockbottom, ditch edges and underwood having dark and moist, scattered light, humus and an altitude of 200-1500 m. Main ingredients of the gynostemma pentaphyllum include saponins, polyphenols, flavonoids, polysaccharides, trace elements such as zinc, selenium, iron, calcium, magnesium and the like, and vitamins A and B. The gynostemma pentaphyllum has the effects of regulating human physiological functions, enhancing human immunity, counteracting side effects of glucocorticoid, resisting oxidation, reducing blood fat and the like.

The gynostemma pentaphyllum has many different species and hybrids (domesticated from wild species first), and also has different ploidy bodies (2×, 4×, 6× . . . ) in natural world. Among different species and different ploidy bodies, bioactive functions and active ingredients have very great differences. However, differences in different places of origin are also great, and particularly the saponins, polyphenols, flavonoids, the trace element selenium and the like have great differences. According to analysis in a laboratory of America, the active ingredients of different gynostemma pentaphyllum produced in different regions in China and in the world have great differences, and any single gynostemma pentaphyllum is the best in all the main active ingredients. In all analysis samples, content of the polyphenols and flavonoids analyzed from diploid gynostemma pentaphyllum in Gynostemma Pentaphyllum Research Institute in Pingli County of Shaanxi is the highest, while content of the saponins and selenium of tetraploid gynostemma pentaphyllum in the institute is the best in all detection samples, and content of total saponins averagely reaches 8.5%. Moreover, in different parts of plants, regardless of diploid or tetraploid, active ingredients of plant leaves are the most ideal. A reasonable gynostemma pentaphyllum formula (2×+4×) contains rich bioactive substances such as polyphenols, flavonoids (particularly rutin and quercetin), saponins, selenium and other substances, and can effectively regulate human normal metabolism, promote human health and lower cholesterol to achieve effects of lowering lipid, softening blood vessels and promoting cardiovascular functions, thereby achieving an effect of regulating blood pressure. However, the active ingredients of the gynostemma pentaphyllum can achieve a certain effect of regulating blood glucose.

In the present disclosure, based on years of analysis and research and cooperative study with University of Maryland, various kinds of gynostemma pentaphyllum in various regions are subjected to systematic and detailed analysis, detection and study. A formula taking single gynostemma pentaphyllum as a main body over the years and a thought of only paying attention to gypenoside are completely changed. A novel composition in the present invention starts from omni-directionally and fully integrating saponins, polyphenol compounds, flavonoid compounds, mineral substances (particularly selenium), and polysaccharide bioactive substances of the gynostemma pentaphyllum. Gynostemma pentaphyllum diploid and tetraploid materials of specific origins and specific species are combined into a brand new gynostemma pentaphyllum formula according to a ratio.

According to bioactive ingredients detected in the present invention, in compounding selenium-enriched health care medicinal materials, an entire effect of regulating the three hypers is further enhanced. If often taken, the composition not only can regulate the three hypers and promote health of internal organs of the body, but also has no any side effect.

Folium eucommiae has the same effects as eucommia ulmoides bark, and is a good medicine for strengthening internal organs since antiquity. Multiple flavonoids and multiple phenolic acids enriched in the folium eucommiae can effectively promote cardiovascular health and coronary blood-flow volume. Moreover, multiple (about 27) lignans compounds are detected. Particularly, a pinoresinol diglucoside compound has an excellent effect of regulating blood pressure.

Alkaloid and folium mori polysaccharide in folium mori can promote activities of insulin β cells; particularly peculiar DNJ (1-deoxynojirimycin) in folium mori is a strong glucose metabolic enzyme inhibitor and can effectively improve insulin resistance, while multiple flavonoids in the folium mori have effects of regulating the three hypers.

Pueraria mirifica grows for many years in selenium-rich soil, has rich selenium content, is enriched in isoflavone, is used for yin deficiency consumptive thirst, spleen-deficiency diarrhea, yang raising and diarrhea checking and liver protecting and stomach nourishing, has an excellent effect of regulating a blood vascular system, and effects in aspects of dilating coronary artery, lowering lipid, reducing blood pressure, regulating blood glucose and the like.

Semen plantaginis is enriched in multiple organic acids, choline, glycoside and polyphenols and has a good diuretic effect, while herba lophatheri contains various flavonoids, bioactive polysaccharides, other phenolic acids and other compounds and also has an excellent effect of inducing diuresis for treating strangurtia.

Pelargonium graveolens is also called rose geranium and originates in southern Africa. The pelargonium graveolens serving as coriander, spice and a medicinal crop is generally cultivated around the world, and is also a horticultural plant with high ornamental value. The pelargonium graveolens is aromatic, has a wide application while serving as a heath care plant, and has a long use history around the world. The medicinal material can achieve effects of helping liver and kidney to eliminate toxins, promoting functions of a lymphatic system, inducing diuresis and helping to relieve stress. Therefore, the pelargonium graveolens is an ideal compatible material for preventing and treating gout.

Sanguisorba minor is also called garden burnet. The sanguisorba minor gains the name since beautiful pinnate leaves are similar to those of elm. The sanguisorba minor originates in north Africa, Europe and central and western Asia. Due to enriched vitamins and refreshing taste, the sanguisorba minor is widely eaten and drunk in Europe and America, e.g., tossing a salad, added into sandwich and juiced to drink. However, due to excellent effects of nourishing and inducing diuresis, the sanguisorba minor serves as a health care supplement for preventing gout locally. In the present formula, when the sanguisorba minor is compounded with other materials, a synergistic effect can be achieved.

In conclusion, the two kinds of gynostemma pentaphyllum (2×, 4×) above are compounded with the above medicinal material to form a formula of the product. A monarch drug and a ministerial drug in the present formula are complementary to each other, and the formula has an effect of regulating self-metabolism of the body, also accelerates education of high uric acid from the body, prevents and treats the traditional “three hypers” and can also effectively prevent and treat hyperuricemia and treat gout. The composition can achieve effects of promoting normal metabolism of the human body, regulating human cell vitality and immunity and strengthening the internal organs of the body, thereby achieving health care effects of effectively preventing and treating “three hypers” and hyperuricemia and enhancing human health and longevity.

In order to achieve the above purposes, a technical solution is adopted in the present invention as follows:

A gynostemma pentaphyllum-containing health care pharmaceutical composition for preventing hyperlipidemia, hypertension, hyperglycemia and gout is characterized by being prepared by mixing raw materials of gynostemma pentaphyllum, folium eucommiae, folium mori, semen plantaginis, herba lophatheri, pueraria mirifica powder, pelargonium graveolens and sanguisorba minor, wherein the raw materials are in percentage by mass: 40-70% of gynostemma pentaphyllum, 5-20% of folium eucommiae, 5-20% of folium mori, 5-10% of semen plantaginis, 2-10% of herba lophatheri, 2-10% of pueraria mirifica powder, 2-8% of pelargonium graveolens and 2-8% of sanguisorba minor.

Further, the pharmaceutical composition is prepared by mixing the following raw materials in percentage by mass: 50-70% of gynostemma pentaphyllum, 6-12% of folium eucommiae, 6-12% of folium mori, 6-12% of semen plantaginis, 6-8% of herba lophatheri. 6-8% of pueraria mirifica powder, 2-5% of pelargonium graveolens and 2-5% of sanguisorba minor.

Further, the pharmaceutical composition is prepared by mixing the following raw materials in percentage by mass: 60% of gynostemma pentaphyllum, 10% of folium eucommiae, 10% of folium mori, 4% of semen plantaginis, 4% of herba lophatheri, 4% of pueraria mirifica powder, 4% of pelargonium graveolens and 4% of sanguisorba minor.

Further, the gynostemma pentaphyllum is prepared by mixing diploid gynostemma pentaphyllum leaves and tetraploid gynostemma pentaphyllum leaves.

Further, a mass ratio of the diploid gynostemma pentaphyllum leaves to the tetraploid gynostemma pentaphyllum leaves is 2:5 to 5:2. Preferably, the mass ratio of the diploid gynostemma pentaphyllum leaves to the tetraploid gynostemma pentaphyllum leaves is 1:1.

Further, the pharmaceutical composition may be prepared into tablets, pills, dissolved medicines, granules and powder.

In the present invention, by compounding the gynostemma pentaphyllum with the traditional Chinese medicines such as folium mori, folium eucommiae and pueraria mirifica powder, dirt, wastes and toxins on inner walls of blood are removed, so that the blood is unobstructed in blood vessels, and blood vessels of the heart, brain, kidney, liver and other organs may be softened; and therefore senescent cells are eliminated from the human body, and self-regulation of the human body restores to be normal. A reasonable gynostemma pentaphyllum formula (2×+4×) can effectively lower the cholesterol to achieve effects of lowering lipid, softening blood vessels and promoting cardiovascular functions, thereby achieving an effect of regulating blood pressure. Moreover, the active ingredients of the gynostemma pentaphyllum formula can achieve a certain effect of regulating the blood glucose.

In the present invention, the diploid gynostemma pentaphyllum and the tetraploid gynostemma pentaphyllum are compounded with domestic and foreign health-care medicinal materials such as folium mori, folium eucommiae, semen plantaginis, herba lophatheri, pueraria mirifica powder, pelargonium graveolens, sanguisorba minor and the like, and a self-regulation function of human health may be effectively promoted, thereby achieving effects of dredging blood vessels, getting rid of the stale and taking in the fresh, strengthening internal organs and lowering “four hypers”. The formula is totally prepared by compounding vegetation medicinal materials with healthy and tonic effects. Compared with a Western medicine, the formula is low in cost and zero in side effect, and can provide many nutritive materials beneficial to health such as saponins, polyphenols, flavonoids, polysaccharides, mineral elements and the like, so that the human body is more healthy.

The compounding also comprehensively considers a theory of traditional Chinese medicinal materials such as four properties and five flavors of traditional Chinese medicinal materials, channel tropism and the like for reference of the compounding. In combination with analysis of effective active ingredients of the used materials, different types of active ingredients (such as saponins, polyphenols, flavones, flavonoids, isoflavones, alkaloids, polysaccharides, organic acids, mineral substances, vitamins and the like) are reasonably combined, and properties and characteristics of different materials are taken into account. In combination with domestic and foreign achievements in scientific research in many years, the formula is prepared. Compared with a conventional drug, the present invention has unique features as follows: starting from sources of “four hypers”, by means of reasonable compounding of multiple medicinal materials according to different ratios, the composition has the effects of promoting metabolism and self-regulation of human cells, reducing blood fat, promoting blood circulation, strengthening the internal organs, promoting a balance, improving biological activity of a blood vascular system, softening the blood vessels, eliminating hidden danger, increasing permeability, inducing diuresis for treating strangurtia and inhibiting the “four hypers”. The problem of “four hypers” is gradually fundamentally solved, and the purposes of rehabilitation and health are finally achieved.

### DETAILED DESCRIPTION OF THE EMBODIMENTS

The present invention is further described below in combination with embodiments. Percentages in the embodiments are all weight percentages, but the present disclosure is not limited to this.

### Embodiment 1

30% of diploid gynostemma pentaphyllum, 30% of tetraploid gynostemma pentaphyllum, 10% of folium eucommiae, 10% of folium mori, 4% of semen plantaginis, 4% of herba lophatheri, 4% of pueraria mirifica powder, 4% of pelargonium graveolens and 4% of sanguisorba minor.

### Embodiment 2

20% of diploid gynostemma pentaphyllum, 50% of tetraploid gynostemma pentaphyllum, 5% of folium eucommiae, 5% of folium mori, 10% of semen plantaginis, 2% of herba lophatheri, 2% of pueraria mirifica powder, 2% of pelargonium graveolens and 4% of sanguisorba minor.

### Embodiment 3

50% of diploid gynostemma pentaphyllum, 20% of tetraploid gynostemma pentaphyllum, 6% of folium eucommiae, 5% of folium mori, 6% of semen plantaginis, 6% of herba lophatheri, 3% of pueraria mirifica powder, 2% of pelargonium graveolens and 2% of sanguisorba minor.

### Embodiment 4

25% of diploid gynostemma pentaphyllum, 25% of tetraploid gynostemma pentaphyllum, 12% of folium eucommiae, 12% of folium mori, 5% of semen plantaginis, 6% of herba lophatheri, 5% of pueraria mirifica powder, 5% of pelargonium graveolens and 5% of sanguisorba minor.

### Embodiment 5

20% of diploid gynostemma pentaphyllum, 20% of tetraploid gynostemma pentaphyllum, 15% of folium eucommiae, 15% of folium mori, 10% of semen plantaginis, 8% of herba lophatheri, 5% of pueraria mirifica powder, 5% of pelargonium graveolens and 2% of sanguisorba minor.

### Embodiment 6

15% of diploid gynostemma pentaphyllum, 30% of tetraploid gynostemma pentaphyllum, 18% of folium eucommiae, 15% of folium mori, 6% of semen plantaginis, 6% of herba lophatheri, 3% of pueraria mirifica powder, 2% of pelargonium graveolens and 5% of sanguisorba minor.

### Embodiment 7

30% of diploid gynostemma pentaphyllum, 15% of tetraploid gynostemma pentaphyllum, 15% of folium eucommiae, 15% of folium mori, 8% of semen plantaginis, 8% of herba lophatheri, 5% of pueraria mirifica powder, 2% of pelargonium graveolens and 2% of sanguisorba minor.

### Embodiment 8

20% of diploid gynostemma pentaphyllum, 20% of tetraploid gynostemma pentaphyllum, 15% of folium eucommiae, 10% of folium mori, 9% of semen plantaginis, 5% of herba lophatheri, 5% of pueraria mirifica powder, 8% of pelargonium graveolens and 8% of sanguisorba minor.

### Embodiment 9 Clinical Embodiments of a Compound Preparation in the Present Disclosure for Treating Gouty Arthritis

Gout belongs to the category of “arthralgia”, and is also called joint-running wind, severe and migratory arthralgia, white tiger joint running and the like. Modern medicine thinks that, the gout is a group of heterogeneous chronic metabolic diseases caused by purine metabolism disorders, and may be divided into an asymptomatic hyperuricemia phase, an acute attack phase of gouty arthritis, an intercritical phase and a chronic phase.

In recent years, along with booming development of traditional Chinese medicine, on the basis of inheriting traditional therapy, traditional Chinese medicine treatment researches of the gout are combined with modern medical experiments, and there are many treatment methods such as internal treatment, external treatment, internal and external combined treatment and the like.

9.1 Data and Methods

60 patients suffering from warm-heat pathogen stagnancy type acute gouty arthritis after clinical diagnosis are selected as research objects. The patients include 36 male patients and 24 female patients, are aged at 51-72, and have an average age of 62±1.2.

With reference to Guiding principles for Clinical Research of New Drugs of Traditional Chinese Medicine and Criteria of Diagnosis and Therapeutic Effect of Internal Diseases and Syndromes in Traditional Chinese Medicine in State Administration of Traditional Chinese Medicine, clinical manifestations of warm-heat pathogen stagnancy type patients suffering from gout are as follows:

Primary symptoms: involved joint burning, red and swollen, and pain from acute onset, and inconvenient flexing and stretching of joints.

Secondary symptoms: fever, tiredness, expectoration or bitter taste, loose stool discomfort or inhibited defecation, and short and yellow urinating.

Tongue vein: reddened tongue, yellow and greasy tongue, soggy pulse or slippery pulse or slip.

The patients should eat low-calorie, low-protein and low-purine foods, strictly withdraw alcohol and particularly beer, drink fructose-containing beverages and yogurt as less as possible, and keep daily water intake at least 2500 mL; the patients should get more rest, keep joint immobilization as much as possible, elevate the injured limb, avoid a heavy load and wear comfortable shoes to avoid the injury of joints; the patients should avoid overfatigue, cold or damp, mental stress and the like, and take medicines that influence uric acid excretion with caution. The compound preparation in the present invention is taken for three times per day at a dosage of 3 g each time. A course of treatment lasts 10 days, and the patients should be continuously treated within 2 courses of treatment.

9.2 Observation Indicators

Blood urea nitrogen (BUN), serum creatinine (Cr), alanine aminotransferase (ALT), aspartate amino transferase (AST) and other hepatorenal functions before and after treatment.

9.3 Evaluation of Therapeutic Effects

Evaluation of clinical therapeutic effects includes determination of therapeutic effects of syndromes of traditional Chinese medicine and determination of clinical comprehensive therapeutic effects. A criterion of the determination of therapeutic effects of syndromes of traditional Chinese medicine is set with reference to Criteria of Diagnosis and Therapeutic Effect of Syndromes in Traditional Chinese Medicine in State Administration of Traditional Chinese Medicine; and a criterion of determination of clinical comprehensive therapeutic effects is drawn up with reference to Guiding principles for Clinical Research of New Drugs of Traditional Chinese Medicine.

Clinical control: clinical symptoms and signs disappear or basically disappear, and a syndrome integral is decreased.

Excellent: the clinical symptoms and signs are obviously improved, and the syndrome integral is decreased.

Effective: the clinical symptoms and signs are improved, and the syndrome integral is decreased.

Ineffective: the clinical symptoms and signs are not obviously improved, or even worsened, and a decrease of the syndrome integral is insufficient.

9.4 Therapeutic Results

TABLE 1
Present attack course of patients

| n | ≤12 hours | ≤1 day | ≤3 days | ≥3 days |
|---|---|---|---|---|
| 40 | 4 | 25 | 8 | 3 |

TABLE 2
Comparison of inflammatory marks of patients
before and after treatment (X ± S)

| Item | Before treatment (n = 40) | After treatment (n = 40) |
|---|---|---|
| WBC (*109/L) | 9.94 ± 1.23 | 4.93 ± 1.20 |
| CRP (mg/L) | 22.36 ± 7.92 | 7.70 ± 0.26 |
| ESR (mm/H) | 38.06 ± 0.24 | 19.24 ± 6.52 |

9.5 Adverse Effects and Treatment:

(1) Influences on hepatorenal functions: after the present research and treatment solution is applied, a treatment group and a control group do not have any obvious abnormal change in detection of the hepatorenal functions such as BUN, Cr, ALT, AST and the like.

(2) Influences on digestive tract functions: after the present research and treatment solution is applied, some cases in the treatment group have an increase of defection frequency and relatively loose stool, but these symptoms are tolerable.

### Embodiment 10

Products in embodiments 1-8 of the present invention are detected according to a national selenium-rich food standard “GB28050-2011”. Detection results show that selenium content is 0.20-5.00 mg/kg and meets the selenium-rich food standard. When detected by an improved new technology based on High Performance Liquid Chromatography (HPLC), the products contain 108.4-188.2 mg/g of saponins, 23.5-37.5 mg/g of polyphenols and 22.5-36.5 mg/g of general flavones. Although the present disclosure is described with reference to specific embodiments above, those of ordinary skill in the art should understand that, the above embodiments are only used for illustrating the present invention, rather than limiting the present invention. Changes and variations made to the above embodiments within the essential spirit of the present invention should be included in the protection scope of appended claims.

We claim:

1. A gynostemma pentaphyllum-containing health care pharmaceutical composition for preventing hyperlipidemia, hypertension, hyperglycemia and gout, prepared by mixing raw materials of gynostemma pentaphyllum, folium eucommiae, folium mori, semen plantaginis, herba lophatheri, pueraria mirifica powder, pelargonium graveolens and sanguisorba minor, wherein the raw materials are in percentage by mass: 40-70% of gynostemma pentaphyllum, 5-20% of folium eucommiae, 5-20% of folium mori, 5-10% of semen plantaginis, 2-10% of herba lophatheri, 2-10%0/of pueraria mirifica powder, 2-8% of pelargonium graveolens and 2-8% of sanguisorba minor.

2. The gynostemma pentaphyllum-containing health care pharmaceutical composition for preventing hyperlipidemia, hypertension, hyperglycemia and gout according to claim 1, wherein the pharmaceutical composition is prepared by mixing the following raw materials in percentage by mass: 50-70% of gynostemma pentaphyllum, 6-12% of folium eucommiae, 6-12% of folium mori, 6-12% of semen plantaginis, 6-8% of herba lophatheri, 6-8% of pueraria mirifica powder, 2-5% of pelargonium graveolens and 2-5% of sanguisorba minor.

3. The gynostemma pentaphyllum-containing health care pharmaceutical composition for preventing hyperlipidemia, hypertension, hyperglycemia and gout according to claim 2, wherein the pharmaceutical composition is prepared by mixing the following raw materials in percentage by mass: 60% of gynostemma pentaphyllum, 10% of folium eucommiae, 10% of folium mori, 4% of semen plantaginis, 4% of herba lophatheri, 4% of pueraria mirifica powder, 4% of pelargonium graveolens and 4% of sanguisorba minor.

4. The gynostemma pentaphyllum-containing health care pharmaceutical composition for preventing hyperlipidemia, hypertension, hyperglycemia and gout according to claim 1, wherein the gynostemma pentaphyllum is prepared by mixing diploid gynostemma pentaphyllum leaves and tetraploid gynostemma pentaphyllum leaves.

5. The gynostemma pentaphyllum-containing health care pharmaceutical composition for preventing hyperlipidemia, hypertension, hyperglycemia and gout according to claim 4, wherein a mass ratio of the diploid gynostemma pentaphyllum leaves to the tetraploid gynostemma pentaphyllum leaves is 2:5 to 5:2.

6. The gynostemma pentaphyllum-containing health care pharmaceutical composition for preventing hyperlipidemia, hypertension, hyperglycemia and gout according to claim 5, wherein the mass ratio of the diploid gynostemma pentaphyllum leaves to the tetraploid gynostemma pentaphyllum leaves is 1:1.

7. The gynostemma pentaphyllum-containing health care pharmaceutical composition for preventing hyperlipidemia, hypertension, hyperglycemia and gout according to claim 1, wherein the pharmaceutical composition is prepared into tablets, pills, dissolved medicines, granules and powder.

8. An application of the gynostemma pentaphyllum-containing health care pharmaceutical composition for preventing hyperlipidemia, hypertension, hyperglycemia and gout according to claim 1, wherein the pharmaceutical composition is used for preventing and treating hyperlipidemia, hypertension, hyperglycemia and hyperuricemia.
